(19) United States
(12) Patent Application Publication
(10) Pub. No.: US 2019/0159675 A1
(43) Pub. Date: May 30, 2019

(54) POINT-OF-CARE TELE MONITORING DEVICE FOR NEUROLOGICAL DISORDERS AND NEUROVASCULAR DISEASES AND SYSTEM AND METHOD THEREOF

(71) Applicants: Rajib Sengupta, Kolkata (IN); Abhijit Das, Kolkata (IN); Anirban Datta, Kolkata (IN)

(72) Inventors: Rajib Sengupta, Kolkata (IN); Abhijit Das, Kolkata (IN); Anirban Datta, Kolkata (IN)

(21) Appl. No.: 16/092,779

(22) PCT Filed: Apr. 12, 2017
(86) PCT No.: PCT/IN2017/050137

§ 371 Date: Oct. 11, 2018

(30) Foreign Application Priority Data
Apr. 13, 2016 (IN) 201631012963

(51) Int. Cl. A61B 5/00; A61B 5/0476; A61B 5/16; A61B 5/1455

(52) U.S. Cl. CPC A61B 5/0022; A61B 5/7435; A61B 5/0006; A61B 5/0024; A61B 5/0476; A61B 5/4064; A61B 5/7405; A61B 5/7264; A61B 5/165; A61B 5/14553; A61B 5/6814; A61B 5/0075

(57) ABSTRACT

Disclosed herein an improved systems and methods using point-of-care (POC), IoT (Internet of Things) enabled device(s) which captures different bio-signals simultaneously as distinct signals, by targeting same neurovascular substrate. The synchronized streaming of the data for live analysis or recording in the tele neuro-monitoring platform are jointly processed in an Artificial Intelligence (AI) based big-data platform under a closed loop, bi-directional, decision tree based system for brain/neurological function status monitoring (continuously and/or intermittently) leading to online POC diagnosis, severity classification, and prognosis of neurological disorders and neurovascular diseases.

### FIELD OF THE INVENTION

This invention, in general relates to a field of medical devices. In particular, the present invention is directed to an improved systems and methods using point-of-care device(s) for determining neuro-glial-vascular interactions and/or monitoring brain/neurological function status leading to diagnosis, severity classification, and prognosis of neurological disorders and neurovascular diseases.

### BACKGROUND OF THE INVENTION

Neurological emergencies are the leading causes of death and disability throughout the world and meeting the urgent healthcare needs of rural patients, especially in developing countries, where they lack adequate neuroimaging facilities, are extremely challenging. There is continuous need to develop a system and method, which is portable and easy-to-use, to provide urgent neuromonitoring to the patients at the point-of-care and in confidence for the emergency and trauma services.

Various approaches have been disclosed in the prior arts in relation to develop devices and systems to answer such needs.

US publication no US20110144520A1 titled Method and device for point-of-care neuro-assessment and treatment guidance discloses a method and apparatus for providing an objective assessment of the neurological state of a patient using a field-portable neuro-assessment device is described. The method includes placing an electrode set on the patient's head, acquiring spontaneous brain electrical signals and evoked potential signals from the patient through the electrode set, processing the signals using a handheld base unit, and displaying a result indicating the probability of the patient's neurological signal being normal or abnormal. The neuro-assessment device allows for a rapid, on-site neurological evaluation by an emergency medical technician, triage nurse, or any other medical personnel to identify patients with neurological disorders who may require immediate medical attention.

U.S. Pat. No. 8,938,301B2 titled Headgear with displaceable sensors for electrophysiology measurement and training discloses a method and system provides for headgear usable for electrophysiological data collection and analysis and neurostimulation/neuromodulation or brain computer interface for clinical, peak performance, or neurogaming and neuromodulation applications. The headgear utilizes dry sensor technology as well as connection points for adjustable placement of the bi-directional sensors for the recoding of electrophysiology from the user and delivery of current to the sensors intended to improve or alter electrophysiology parameters. The headgear allows for recording electrophysiological data and biofeedback directly to the patient via the sensors, as well as provides low intensity current or electromagnetic field to the user. The headgear can further include auditory, visual components for immersive neurogaming. The headgear may further communication with local or network processing devices based on neurofeedback and biofeedback and immersive environment experience with balance and movement sensor data input.

US publication no 20110087125A1 titled System and method for pain monitoring at the point-of-care discloses a method and apparatus for providing objective assessment of pain using a field portable device is described. The method includes placing an electrode set coupled to a handheld base unit on the subject's head, acquiring brain and/or peripheral nervous system electrical signals from the subject through the electrode set, processing the acquired brain electrical signals using a feature extraction algorithm stored in a memory of the base unit, classifying the processed signals into pain categories, determining an objective quantification of the pain level, and indicating the pain category and/or pain scale on the handheld base unit. The memory of the base unit stores a reference database for classification of the processed signals, or the base unit is configured to wirelessly access the reference database from a remote data storage unit.

U.S. Pat. No. 9,510,765B2 titled Detection and feedback of information associated with executive function discloses a neurosensing and feedback device to detect mental states and alert the wearer, such as in real-time. In an example, neural activity is detected by sensors that measure frequency, amplitude, synchrony, sequence and site of brain activity. These measurements can be compared to neural signatures and patterns shown to be correlated to neuropsychological conditions and disorders. When these measurements indicate an undesirable state the wearer is alerted via visual, audible or tactile means designed to be highly effective at alerting the wearer and allowing them to adjust their brain activity. Executive function, known to be crucial for school readiness, academic achievement and successful life outcomes, is the chief state to be detected, trained and supported. The device is designed to be used during primary activities, e.g. reading and listening, and to not require third party intervention during primary use.

EP no 3064130A1 titled Brain activity measurement and feedback system discloses a head set (2) comprises a brain electrical activity (EEG) sensing device (3) comprising EEG sensors (22) configured to be mounted on a head of a wearer so as to position the EEG sensors (22) at selected positions of interest over the wearers scalp, the EEG sensing device comprising a sensor support (4) and a flexible circuit (6) assembled to the sensor support. The sensor support and flexible circuit comprise a central stem (4a, 6a) configured to extend along a center plane of the top of the head in a direction from a nose to a centre of the back of a wearers head, a front lateral branch (4b, 6b) configured to extend across a front portion of a wearer's head extending laterally from the central stem, a center lateral branch (4c, 6c) configured to extend across a top portion of a wearer's head essentially between the wearer's ears, and a rear lateral branch (4d, 6d) configured to extend across a back portion of a wearer's head.

US publication 20140303424A1 titled Methods and systems for diagnosis and treatment of neural diseases and disorders discloses methods and systems for modulating activity of a nervous system component. Neural pattern recognition is used to identify a neurological and/or psychiatric disease or disorder based on input generated by electric signals indicative of the subject's brain activity. In an embodiment, the method comprises receiving an input from one or more sensors, each sensor configured to sense a particular characteristic indicative of a neurological or psychiatric condition or state; developing treatment parameters based on the input received from the one or more sensors; and generating neural modulation signals for delivery to a nervous system component through one or more output devices in accordance with one or more developed treatment parameters.

Chinese application CN202161317U titled Helm for acquiring brain signal by combining electroencephalography and near-infrared spectroscopy discloses a utility model relates to a helm for acquiring a brain signal simultaneously by organically combining electroencephalography (EEG) and near-infrared spectroscopy. An EEG electrode and a near-infrared probe holder are fixed on a flexible material covering the scalp; a near-infrared probe is coupled with the near-infrared probe holder; the near-infrared probe holder consists of a near-infrared probe holder A and a near-infrared probe holder B; the near-infrared probe consists of a near-infrared probe A for emitting infrared rays and a near-infrared probe B for receiving the infrared rays; the near-infrared probe holder A is used for fixing the near-infrared probe A; the near-infrared probe holder B is used for fixing the near-infrared probe B; the near-infrared probe holder A and the near-infrared probe holder B are distributed on two sides of each EEG electrode; and the space between the near-infrared probe holder A and the EEG electrode is equal to that between the near-infrared probe holder B and the EEG electrode. The helm acquires blood oxygen change information in the region of 2 to 3 cm below the EEG electrode, so that a researcher can know the change condition of the brain function in all dimensions and new power is provided for brain function recognition and brain-control robot research.

PCT publication WO2011135136A1 titled Device for stimulating the nervous system using a static magnetic field and use of such a device discloses a device for different neurological, psychiatric and central-nervous-system disorders. Invasive and non-invasive embodiments are described. The non-invasive embodiment comprises magnets (12) held by a supporting element (14, 15) that can be adjusted to fit the user's head, means (16) for attaching the magnets to the supporting element (14, 15), means (30) for separating the magnets from the user's scalp, and a power source (32) for the separating means (30). The invasive embodiment comprises magnets (12), a supporting element (14, 15) and means (16) for attaching the magnets to the supporting element (14, 15). The supporting element (14, 15) may be a cap (14) or a helmet (15).

An article titled “Robust pre-clinical software system for real time NIRS and EEG monitoring” published in December 2014 in the publication ‘Polytechnic School Of Montreal’ wherein it is disclosed a design and implementation of a real-time software system to support a bimodal NIRS and EEG brain imaging device. Real-time information on brain activity is an important factor in early detection and diagnosis at the top level of the cortex of various brain disorders. Current software systems provide limited real-time parameter adjustment and automated features for quick and easy analysis. The project presented in this master's thesis is part of the multidisciplinary IMAGINC research group, with the objective of developing a wireless, non-invasive and portable brain imaging system that allows imaging of the whole cortex in real time. The hardware system is capable of recording data from 128 NIRS and 32 EEG channels, as well as additional accelerometer and analog channels through the optodes and electrodes mounted onto the helmet. The software system acquires the real-time data from the hardware module using a wireless connection and displays the hemodynamic variations on the user interface. The change in hemodynamic activity is displayed on a 2D map of the brain, with selection of different views. Remote monitoring is also possible since the data can be transferred wirelessly to another computer. Through the user-friendly and intuitive user interface, the user can control and adjust various test parameters throughout the acquisition without any interruption. In order to achieve maximum illumination setting for individual subjects there is an automatic calibration function that quickly adjusts the illumination intensity for each of the emitters in just a few seconds. Previously defined NIRS and EEG configuration files (bipolar and referential montage) can be uploaded for easy testing. An automated analysis feature quickly analyzes and reports the status of all NIRS channels during the test to ensure good connection and valid results. The designed system can successfully record and process data for a continuous period of up to 24 hours. The results have been validated using similar NIRS data analysis software during figure tapping tasks and the hemodynamic variations were as expected.

However, currently available solutions as disclosed above have their own restrictions in integrating the various noninvasive tools and enable them functioning simultaneously and provide effective point-of-care continuous bedside monitoring of deleterious effects of brain trauma.

It would be desirable, therefore, to provide point-of-care system and method which is noninvasive and enable immediate and effective monitoring and guiding to the patients.

### SUMMARY OF THE INVENTION

The present invention relates to an apparatus for the telemonitoring of neurovascular coupling by the recordal of neural and haemodynamic responses. More particularly this invention pertains to a novel apparatus capable of ascertaining neurovascular coupling wherein multiple bio-signals (NIRS/EEG/EOG/ECG/PPG/BLOOD-PRESSURE) from brain and or body is captured simultaneously as distinct signals in point-of-care IoT (Internet of Things) enabled device(s), and simultaneously streamed to a tele neuro-monitoring platform in a time synchronized manner, where they are jointly processed in an Artificial Intelligence (AI) based big-data platform under a closed loop, bi-directional, decision tree based system for brain/neurological status monitoring. The present invention has overcome quite a few time-consuming procedures and devices of the prior art and this will allow remote, point-of-care and automatic diagnosis, severity classification and prognosis of neurological diseases and disorders.

It is an objective of the present invention to provide a robust point-of-care IoT based continuous bedside neuromonitoring device for uses during the transfer of the patient to clinic such that the patients can receive the urgent care they needs immediately and in the confidence at the emergency and trauma services.

It is another objective of the present invention wherein more than one bio-signal and respective sensor technologies such as multi-wavelength optical (NIRS—near-infrared spectroscopy as well as extended near infrared spectroscopy from ˜700 nm to 2500 nm) and electrophysiological (EEG—Electroencephalography) is used simultaneously to target same neural substrate in a single sensor montage using beam-forming approaches, which enables effective point of care continuous bedside monitoring of the neurological disorders and neurovascular diseases.

In accordance with one preferred embodiment of the present invention, there is provided a closed-loop, bi-directional, decision-tree based Point of Care (POC) system and method that provides remote diagnosis of a patient's medical condition, the system comprising a POC device that includes a component (head mountable and/or attached with body parts) with one or more sensors capable to acquire bio-signals from brain and/or body, an electronics/electrical component of the device configured to capture one or more bio-signals from the sensors with system for synchronizing streaming data for live analysis or recording, an IoT (Internet of things) enabled component with a Graphical User Interface (GUI) of the device interacting with the electronics/electrical component wirelessly or in a wired manner to receive the bio-signals, the IoT/GUI component also captures patient's vitals and other pertinent medical information from multiple sources (e.g: Other medical devices, Patient's Personal health Record, Onsite Healthcare Provider's physiological observation of the patient) and transfers the bio-signals along with the patient's medical information (collectively known as “input”) to a tele-monitoring platform, wherein a multi-level, decision-tree based module (Diagnosis module), deployed in the tele-monitoring platform analyzes the “input”, aggregates the output decision at each level of the decision-tree system and provides the output decision as input to next level of the decision-tree system leading towards accurate diagnosis of patient's medical condition.

In accordance with one preferred embodiment of the present invention, there is provided a point of care (POC) system for determining neuro-glial-vascular interaction, wherein the system comprising one or more sensor configured to sense a particular characteristic indicative of a neurological or psychiatric condition or state, means for receiving an input as a bio-signal from one or more sensors and developing treatment parameters based on the input data, a determination means configured to receive bio-signals, specifically EEG and NIRS bio-signals and perform analysis, means for storing and comparing the data relating to NIRS and EEG signal generated by the determination means for comparing the detected abnormalities in NIRS and EEG values with a reference signal values, a display means for displaying a content based in part on the data output from said determination means, wherein the content comprises a signal indicative of the presence or absence and/or severity, a transfer means to transfer the data wirelessly to a remote monitoring center to take the required decision, wherein the system characterized in that utilizes two or more sensor modalities such as multi-wavelength optical and electrophysiological, which are used simultaneously to target same neural substrate in a single sensor montage using beam-forming approaches to enables effective point of care monitoring of the neurological disorders and neurovascular diseases.

In accordance with our preferred embodiment of the present invention to capture neuro-glial-vascular interaction, we will use the complete electromagnetic and spectral landscape of the biological tissue. Here, hemoglobin (oxygenated and deoxygenated hemoglobin) are the dominant endogenous absorbers in the 700-900 nm region, whereas the 900-1400 nm region provides another set of chromophores. Water displays characteristic absorption bands around 970, 1200, and above 1400 nm, while lipids exhibit unique bands at different wavelengths up to 2500 nm. To capture the neurovascular coupling status in the neural tissue, this overall water-lipid-protein spectral profile of the tissue can be considered to an optical signature of the neuro-glial-vascular tissue along with the oxygenated and deoxygenated hemoglobin as the hemodynamic signature, and the EEG as the neural activity signature.

The system disclosed herein the present invention to capture neuro-glial-vascular interaction is having wherein the one or more sensors comprise EEG and NIRS along with other analytical tool, wherein said EEG and NIRS are used simultaneous to detect spreading depolarization in brain trauma affecting neuro-glial-vascular tissue, wherein the simultaneous multi-modality multi-distance recording of EEG and multi-wavelength NIRS during spreading depolarization/depression of spontaneous activity is not only detect Neurovascular Coupling (NVC) dysfunction and assess secondary brain injuries, but also adds to the therapeutic accessibility of the syndrome under a remote human-in-loop triage decision making system.

In accordance with another embodiment of the present invention, there is provided a low-cost robust point of care continuous neuromonitoring device for use during the transfer of the patient to clinic such that the patients receives the urgent care they need immediately and in confidence at the emergency trauma services, wherein said device is characterized by having simultaneous multi-modality multi-distance recording of EEG and multi-wavelength NIRS during spreading depolarization/depression of spontaneous activity is not only detect neurovascular coupling (NVC) dysfunction and assess secondary brain injuries, but also adds to the therapeutic accessibility of the syndrome under a remote human-in-loop triage decision making system.

In accordance with one preferred embodiment of the present invention, there is provided a bi-directional, decision-tree based Point of Care (POC) method, the method comprising receiving input data from one or more sensors, each sensor configured to sense characteristics indicative of neurological conditions of a patient, transferring the data wirelessly to a tele-monitoring center, analyzing the data received from the one or more sensors at the tele-monitoring center, wherein the analysis comprises triggering a multi-level decision tree based on one or more parameters of the patient, aggregating output decisions at each level of the multi-level decision tree, providing one or more aggregated output decisions as input to next level in the multi-level decision tree; and developing treatment parameters based on the aggregated output decisions for accurate diagnosis of patient's medical condition.

In accordance with further embodiment of the present invention, there is provided a point-of-care monitoring (POCT) method for neuro-glial-vascular interactions, wherein the method comprising receiving an input of a neurological or psychiatric condition from one or more sensors and developing treatment parameters based on the input data, determining the signals received from EEG is used with NIRS and comparing the data relating to NIRS and EEG signal for the detected abnormalities values with a reference signal values, further analyzing and transferring the data wirelessly to a remote monitoring center to take the required decision,

Wherein the method characterized in that utilizes two or more sensor modalities such as multi-wavelength optical and electrophysiological, which are used simultaneously to target same neural substrate in a single sensor montage using beam-forming approaches to enables effective point of care monitoring of the neurological disorders and neurovascular diseases.

In accordance with yet another embodiment of the present invention, there is provided a method for neuro-glial-vascular interactions, wherein the communication is bidirectional between the POC device and the remote human as well as software agents working in concert, and wherein point of care (POC) data as well as metadata (observations by paramedic) is relayed by the device client (IoT) to remote telemonitoring center (data server) where it's tagged online for neurovascular dysfunction, and the NIRS-EEG sensor montage is automatically reconfigured at the server side to target that specific (region of interest of the remote human as well as software agent) neural tissue at POC.

The method according to the embodiments, wherein the one or more sensors are enable to sense brain activity by targeting same neural substrate simultaneously and passing the signals to the analyzer and wherein said analyzer analyzes the brain activity for any neurological disorders and neurovascular diseases and wherein the one or more sensors comprise electroencephalography (EEG) and near-infrared spectroscopy (NIRS) along with other analytical tool, wherein said EEG and NIRS are used simultaneous to detect spreading depolarization in brain trauma and thus able to detect presence or absence of Traumatic Brain Injury in its multiple variations affecting neuro-glial-vascular tissue.

In accordance with another embodiment of the present invention, there is provided a method for neuro-glial-vascular interactions, wherein the simultaneous multi modality multi-distance recording of EEG and multi-wavelength NIRS during spreading depolarization/depression of spontaneous activity is not only detect NVC dysfunction and assess secondary brain injuries affecting neuro-glial-vascular tissue, but also adds to the therapeutic accessibility of the syndrome under a remote human-in-loop triage decision making system.

In accordance to another embodiment, the simultaneous multi modality multi-distance recording of EEG and multi-wavelength NIRS during brain hypoxia affecting neuro-glial-vascular tissue allow to detect the presence of ischemic stroke by automated calculation of Delta-Alpha ratio (DAR) derived from quantitative analysis of EEG data and simultaneous measurement of oxy-hemoglobin (HbO2) and de-oxy hemoglobin levels measured from NIRS.

Further, the method provided according to the above embodiments, wherein said method is autoregressive (ARX) method and wherein said method is utilized to capture the coupling relation between regional cerebral haemoglobin oxygen saturation and the log-transformed mean-power time-series for EEG, wherein subject-specific alterations of ARX poles and zeros with different dead time provides relevance for diagnosing neurovascular dysfunction in cerebrovascular occlusive disease affecting neuro-glial-vascular tissue.

The method provided according to the above embodiments, wherein the sensor enabled interaction between the different components used herein in the system such as data is received and spread or bifurcated with Doctor and Data analytics center and wherein said analytics center provides diagnosis and monitoring of neurovascular dysfunction in cerebrovascular occlusive disease of the patient affecting neuro-glial-vascular tissue and allow deliverance of treatment like thrombolysis through administration of various thrombolytic agents like recombinant tissue plasminogen activator and/or mechanical thrombolytic/thrombectomy agents through remote monitoring and guidance.

In another embodiment, the simultaneous recording of NIRS and EEG allow rapid and remote diagnosis and monitoring of hypoxic ischemic encephalopathy affecting neuro-glial-vascular tissue and subsequent brain injury status in children especially neonates. The quantification of the tissue oxygen saturation of hemoglobin (rStO2) through NIRS in percentage from 0 to 100%, where the normal ranges of rStO2 has been estimated to be from 55% to 85%, and the addition of amplitude-integrated EEG/EOG (aEEG) parameters, where aEEG is a trend generated from two channel (C3-P3, C4-P4) EEG recordings that are typically filtered to exclude frequencies other than 2 to 15 Hz and then displayed on a compressed time scale, allow the non-invasive diagnosis and monitoring.

Further, the method provided according to the above embodiments, the real-time brain/neurological-state monitoring provided by NIRS and EEG allow the neuromonitoring during therapeutic hypothermia (TH) which is a standard care in neonatal HIE and also in adult HIE. The apparatus specifically allow diagnosis of electrographic-only seizures or non-convulsive seizures (NCS) affecting neuro-glial-vascular tissue which are common during TH and allow its treatment possible through real-time and possible remote monitoring allowing TH to be applied at point-of-care.

In further embodiment, the apparatus is also be used for diagnosis, often remote, of epileptic emergencies like non-convulsive status epilepticus (NOSE) which are common to many neurological emergencies affecting neuro-glial-vascular tissue. The transfer of EEG data allow real-time automated algorithms to run which distinguish between non-convulsive seizure patterns that occur in acute care settings and other organized rhythmic patterns characteristic of toxic or metabolic encephalopathies. The addition of NIRS parameters allow further sub-classification as differences in cerebral oxygen availability were noted between different types of seizures (e.g., electrographic seizures were accompanied by rapid reductions in HbO2 and cerebral blood volume without reduction of cytox, whereas electroclinical seizures are characterized by marked increases in HbO2 with or without reduction of cytox).

Other embodiments of the invention are disclosed herein. The foregoing and other features, utilities and advantages of various embodiments of the invention will be apparent from the following more particular description of the various embodiments of the invention as illustrated in the accompanying drawings and claims.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIGS. 1(a), (b) and (c) illustrates a high level drawings of exemplary system and method to monitor and determine neuro-glial-vascular dysfunction.

FIG. 2 illustrates a high level system and method diagram of Remote human-in-loop triage decision making system.

FIG. 3 illustrates a high level drawing of a device used for point of care monitoring of neuro-glial-vascular interaction using a cap with shape detection capability, e.g., based on 2D impedance measures. Here, NIRS and EEG signal for either the whole brain or a region of interest as well as corresponding sensor location information are sensed by the Transceiver.

FIG. 4 illustrating (A) inverse neurovascular coupling: reverse neurovascular coupling between spreading depolarizations (SD) and cerebral blood flow and (B) Correlates of SDs in the scalp EEG as slow potential changes (a) and depressions of spontaneous activity (b and c), serving as potential biomarkers in continuous EEG recordings, and (C) the corresponding alterations in regional hemodynamics.

FIG. 5 (A) illustrates digital tapping of anterior temporal artery to capture systemic artefacts using multi-distance measures.

FIG. 5(B) illustrates a NIRS-EEG joint-imaging unit with Long-separation and Short-separation photo-detectors.

FIG. 6 illustrates NIRS-EEG joint-imaging of same brain tissue based on pre-computed sensitivity of the sensors and the cap with shape detection capability, primarily in the, middle frontal and superior frontal gyrus of brodmann area 6 to assess neurovascular coupling

### DETAILED DESCRIPTION OF THE INVENTION

The following detailed description provides further details of the figures and embodiments of the present application. Reference numerals and descriptions of redundant elements between figures are omitted for clarity. Terms used throughout the description are provided as examples and are not intended to be limiting.

It is to be understood that the following disclosure describes several exemplary embodiments for implementing different features, structures, or functions of the invention. Exemplary embodiments of components, arrangements, and configurations are described below to simplify the present disclosure; however, the descriptions of these exemplary embodiments are provided merely as examples and are not intended to limit the scope of the invention.

Additionally, the present disclosure may repeat reference numerals and/or letters in the various exemplary embodiments and across the figures provided herein. This repetition is for the purpose of simplicity and clarity and does not in itself dictate a relationship between the various exemplary embodiments and/or configurations discussed in the various figures. Finally, the description of exemplary embodiments presented below may be combined in any combination, e.g., any element from one exemplary embodiment description may be used in any other exemplary embodiment, without departing from the scope of the disclosure.

In accordance with another aspect of the present invention, there is provided a system, wherein the system is remote human-in-loop triage decision making system comprising a sensors, a trans-receivers, a storage where the data is stored and Data analytics center, wherein the sensor enable interaction between the different components used herein in the system such as data is received and spread or bifurcated with doctor and data analytics center and wherein said analytics center provides diagnosis of neurovascular dysfunction in cerebrovascular occlusive disease of the patient.

According to one of the exemplary embodiment of the present invention, there is provided an autoregressive (ARX) method to capture the coupling relation between regional cerebral haemoglobin oxygen saturation and the log-transformed mean-power time-series for EEG, wherein subject-specific alterations of ARX poles and zeros with different dead time provides relevance for diagnosing neurovascular dysfunction in cerebrovascular occlusive disease. There are various methods that can be used to assess the degree of similarity or shared information between two signals. Some of these methods depend on the type of presumptive system which processes the one “input” signal into the other “output” signal. For linear memory less systems, cross correlation in time domain is used. For linear systems with memory, common methods include autoregressive models with exogenous input (ARX), autoregressive moving average (ARMA) etc. Complex systems such as brain are difficult to analyze because of the huge number of individual neuronal/synaptic paths between nuclei, the nonlinear and non-stationary nature of neuronal connections, and the operation at multiple time scales. One approach is to use simple low order linear models to approximate the transfer function relationship, such as autoregressive with exogenous (ARX) models. The advantage of using linear ARX models is that there is no need to estimate nonlinearity parameters, and less training data is required. However, the performance of such models depends on the model order, scale and pre-filtering. In this study, we adapt and apply the ARX model approach to evaluate the degree of correlation between cortical EEG and oxy haemoglobin dynamics at low frequency oscillations. The ARX model is a common method to represent output signals from an unknown system by using a linear combination of past output signal values and past input values.

In accordance with another aspect of the present invention, there is provided Point-of-care monitoring (POCT) method for neuro-glial-vascular interactions, wherein the method comprises electroencephalography (EEG) is used with Near Infrared spectroscopy (NIRS) for optical monitoring of the cerebral matter to detect the inverse neurovascular coupling in order to prevent secondary brain injury, and wherein the NIRS reflects a complementary hemodynamic signature of spreading depolarization in the study of the relationship between neuronal activity and cerebral haemodynamics affecting neuro-glial-vascular tissue.

According to the present invention, a bi-directional, decision-tree based Point of Care (POC) system is provided wherein the system comprising a point of care (POC) device(s) that includes: component(s) (head mountable and/or attached to body parts) comprising one or more sensors capable to acquire multiple bio-signals from brain and/or body within a body area network, a Detection module configured to receive NIRS and EEG signal for either the whole brain or a region of interest as well as corresponding sensor location information (i.e., sensor montage using cap with shape detection capability), and perform analysis (e.g., ARX method)—an electronics/electrical component(s of the device configured to capture one or more bio-signals from the sensors, a Storage module for locally storing data relating to NIRS and EEG sensors, e.g., sensor location sensitivity values, such that the NIRS and EEG sensors can be re-configured (i.e., change in the sensor montage) to focus on a region of interest for the Detection module, a Processing module for comparing the detected abnormalities in NIRS and EEG values with a reference signal values, e.g., that are stored in the Storage module, as well as to determine the sensor montage to target as queried region of interest according to sensor sensitivities stored in Storage module, a Display module for displaying a content based in part on the data output from said Detection module, wherein the content comprises a signal indicative of the presence or absence and/or severity of the neuro-glial-vascular dysfunction, a Transfer module to bidirectionally transfer the data wirelessly to a remote monitoring center (e.g., over a secure virtual private network) and for synchronizing streaming data for live analysis or recording—an IoT (Internet of things) enabled component with a Graphical User Interface (GUI) of the device interacting with the electronics/electrical component wirelessly or in a wired manner to receive the bio-signals as well as captures patient's vitals and other pertinent medical information from multiple sources (e.g: Other medical devices, Patient's Personal health Record, Onsite Healthcare Provider's physiological observation of the patient) and transfers the bio-signals along with the patient's medical information (collectively known as “input”) to a tele-monitoring platform and a multi-level, decision-tree based diagnosis and triaging (i.e., Diagnosis module), deployed in the tele-monitoring platform analyzes the “input”, aggregates the output decision at each level of the decision tree and provides the output decision as input to next level of the decision-tree system leading towards accurate diagnosis of patient's medical condition.

Further, the POC system disclosed herein for determining neuro-glial-vascular interaction, wherein the system comprising one or more sensor (re)configured to sense a particular characteristic indicative of a neurological or psychiatric condition or state, means for receiving an input from one or more remotely configurable sensors to target a cortical region of interest, and developing treatment parameters based on the input data, a determination means configured to receive NIRS and EEG signal and perform analysis remotely, means for storing and comparing the data relating to NIRS and EEG signal generated by the determination means for comparing the detected abnormalities in NIRS and EEG values with a reference signal values, means for the human and/or software agents to remotely query a specific neural tissue or cortical location (region of interest) from the whole-head montage of EEG and NIRS sensors at the point of care (POC), means of finding the subject specific shape of the cap, e.g., based on the impedance changes in the cap material, and therefore the sensor locations such that the sensor montage can automatically re-configured at the POC device based on pre-computed sensitivity of the sensor locations, a display means for displaying a content based in part on the data output from said determination means, wherein the content comprises a signal indicative of the presence or absence and/or severity and a transfer means to transfer the data wirelessly to a remote monitoring center to take the required decision with the Diagnosis module, wherein the system characterized in that utilizes two or more sensor modalities such as multi-wavelength optical and electrophysiological, which are used simultaneously to target same neural substrate in a single sensor montage using beam-forming approaches to enables effective point of care monitoring of the neurological disorders and neurovascular diseases.

In accordance with another aspect of the present invention, there is provided a system to detect spreading depolarization in brain trauma using simultaneous recording of EEG and NIRS, wherein the system comprising integrating the POCT device for brain trauma monitoring at the Medical Emergency System towards point-of-care sensors with remote human-in-loop triage decision making system using internet of things.

According to the present invention, there is provided a point of care system wherein multiple bio signals from brain is captured in the device and then processed in the tele neuro-monitoring platform under a closed loop, bi-directional, decision tree based system which is further processed using artificial intelligence system aided by machine learning algorithm.

According to the present invention, the low-cost POCT device based on simultaneous multi modality multi-distance recording of EEG and multi-wavelength NIRS during spreading depolarizations/depression of spontaneous activity is not only detect neurovascular coupling (NVC) dysfunction and assess secondary brain injuries, but also add to the therapeutic accessibility of the syndrome under a remote human-in-loop triage decision making system.

According to the present invention, there is provided a tele-health platform which enable interaction between patient and specialist physician remotely—though for better resource utilization, care-provider with increasing level of expertise (such as Community Health Worker<Paramedic<Nurse<General Physician<Pediatrician<Neonatologist (for Neonate and Children)<Neurologist<Neurosurgeon etc.). It is introduced under a closed, bi-directional, decision tree based system running along with artificial intelligence and machine learning algorithms.

According to one of the embodiment, in case of a suspected Stroke, along with the NIRS+EEG combined signal, the onsite care-provider provides patient's medical history including physiological observation which is transmitted from handheld to cloud server. The data is processed and analysed using proprietary algorithms, which is assessed by the care-provider remotely. If needed, the remote care-provider can also use built-in audio-video facilities in the CEREBROS platform (and the handheld) to observe and interact with the patient, thus providing the human-touch that most patients.

According to the present invention the point of care system is being developed as an integrated innovation, consisting of scientific/technological, social and business innovation, for an end-to-end solution for Neurological diseases, specifically for emergency situations.

The current system is providing an end to end solution (360 degree) to the end user, the patient. The intervention is done as locally as possible, as close to patient's home. Upon diagnosis at point-of-care by the system disclosed herein, the patient and the care-provider attending the patient, are transported to the Hospital ensuring that is nearest from patient's location, it is having appropriate resource and facilities and have availability to accommodate the patient.

The system and method provided according to the embodiments disclosed in the present invention is point of care multi-modal and enable real time continuous functioning remotely.

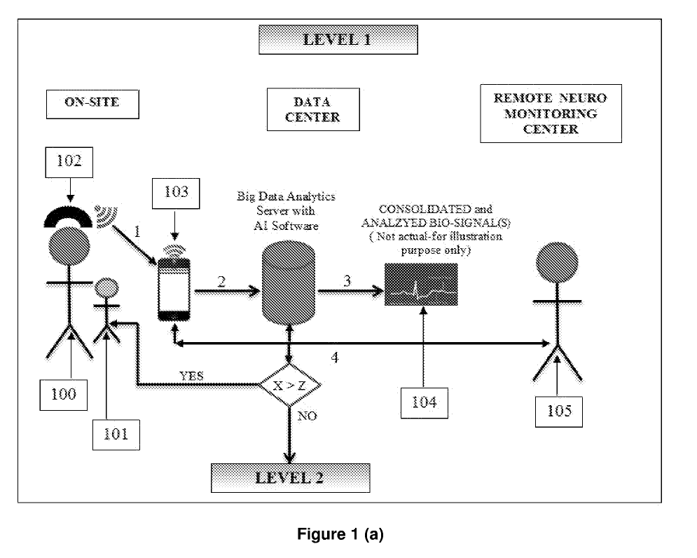

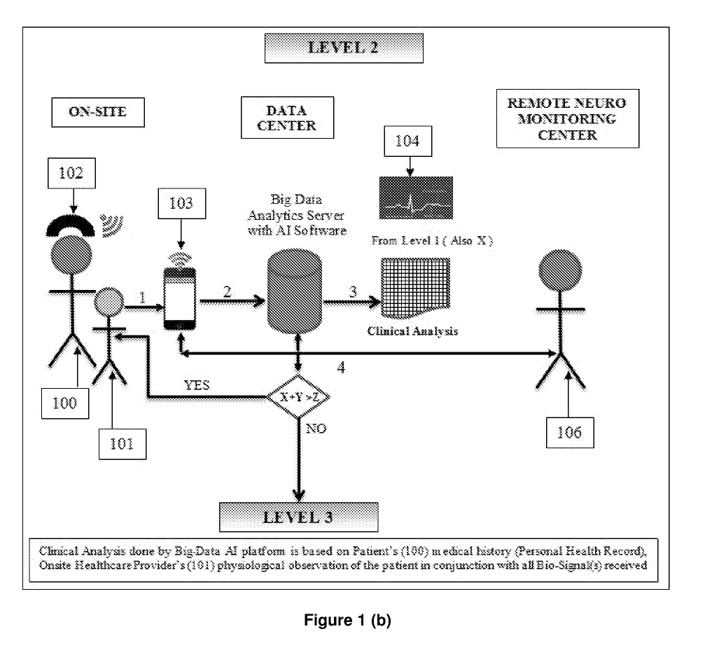

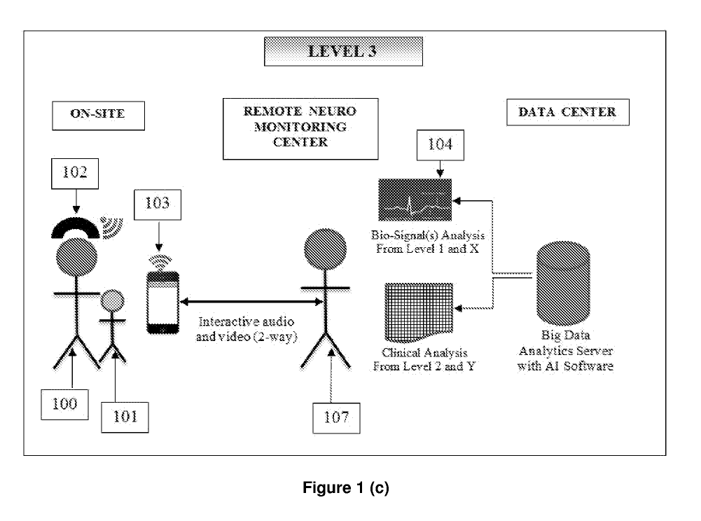

FIG. 1 (a), detailed the Point-of-care (POC) brain/neurological monitoring system and method, wherein a patient (100) suspected with neurovascular disease A, a care seeker (101) who is minimally trained is onsite patient (100), wherein the care seeker uses a POC device (102) on the patient and wherein bio-signals (e.g.: NIRS, EEG) from the sensors component (head mountable and/or attached with body) of the POC device is transferred to the IoT enabled component of the device (103), which transfers the bio-signals (“input”) to the tele-monitoring platform (104), wherein a multi-level, decision-tree based Diagnosis method is triggered analyzing the “input” and providing a report to appropriately trained healthcare professional (Care provider 1) (105). The received report is interpreted and tagged (bio-marked) with X probability of neurovascular disease ‘A’ and send back to ‘Care seeker’ (101) on the GUI component of the POC device (103) as an output from the Level 1 of the Diagnosis module. If X>Z, (Where Z is the pre-determined threshold of neurovascular disease ‘A’), then Patient is diagnosed with the neurovascular disease ‘A’.

If X is NOT>Z, refer FIG. 1 (b), wherein care-seeker (101) is advised to send Patient's (100) clinical information and which introduces Care provider 2 (106) whose clinical skill level>Care Provider 1 (105), wherein the care seeker (101) or other sources (e.g.: vital collecting medical device, Patient's Personal Health record etc) provides patient's medical information including physiological observation appropriate for neurovascular disease ‘A’ from GUI component of the POC device (102) to tele-monitoring platform (104), where the platform processes the input (Patient's medical information+physiological observation+output received from Level 1 of the method detailed in FIG. 1(a)), which is further assessed by Care Provider 2 (106), to provide (X+Y) probability of neurovascular disease ‘A’, which again goes back to the ‘Care Seeker’ (101) via GUI component of the POC device (102), if (X+Y)>Z (Where Z is the pre-determined threshold of neurovascular disease ‘A’), then Patient is diagnosed with the neurovascular disease ‘A’.

If (X+Y) NOT>Z, refer FIG. 1 (c), wherein Care Provider 3 (107) is introduced, who is clinically trained in neurovascular disease A and his/her skill level>Care Provider 2 (106), wherein using bi-directional interactive audio-video, Care provider 3 (107), with the resultant output obtained from the method detailed in FIGS. 1(a) and (b) and findings (NIRS+EEG report, Patient's Medical Information and (X+Y) probability), provides remote diagnosis/screening of neurovascular disease ‘A’ in the Patient (100) to the Care seeker (101) for further clinical management of the Patient (100).

According to one of the exemplary embodiment of the present invention there is provided a multi-level remote diagnosis using the device and the tele-monitoring system, wherein the diagnosis is detailed in the following manner:

Referring the Diagnosis module indicated in the FIG. 1(a), a Patient who is suspected with Neurovascular Disease A, an individual care seeker who is onsite with patient and minimally trained in the device and a care provider; healthcare professional who is minimally trained to interpret bio-signals such as EEG/NIRS signals, wherein the care seeker uses a POC device on the patient and wherein bio-signals (e.g.: NIRS, EEG) from the sensors component (head mountable and/or attached with body) of the POC device is transferred to the IoT enabled component of the device, which transfers the bio-signals (“input”) to the tele-monitoring platform, wherein a multi-level, decision-tree based Diagnosis module is triggered analyzing the “input” and providing a report to appropriately trained healthcare professional (care provider 1). The received report is interpreted and tagged (bio-marked) with X probability of neurovascular disease ‘A’ and sent back to ‘Care seeker’ on the GUI component of the POC device as an output from the Level 1 of the Diagnosis module.

wherein, if X>Z (where Z is the pre-determined threshold of neurovascular disease ‘A’), then Patient is diagnosed with the neurovascular disease ‘A’

if X is NOT<Z,

then care-seeker is advised to send Patient's clinical information as well as Care provider 2, whose clinical skill level>Care Provider 1 is introduced in this Level 2 (refer FIG. 1 (b))

In this Level 2, the care seeker with the patient or other sources (e.g.: vital collecting medical device, Patient's Personal Health record etc) provides patient's medical information including physiological observation appropriate for neurovascular disease ‘A’, from GUI component of the POC device to tele-monitoring platform, wherein the platform processes the input Patient's medical information+physiological observation+output received from Level 1 of the method detailed in FIG. 1(a)), which is assessed by Care Provider 2, to provide (X+Y) probability of neurovascular disease ‘A’ which again goes back to the ‘Care Seeker’ (101) via GUI component of the POC device (103), Wherein if (X+Y)>Z (Where Z is the pre-determined threshold of neurovascular disease ‘A’), then Patient is diagnosed with the neurovascular disease ‘A’

if (X+Y) is NOT<Z, then Care provider 3, who is clinically trained in neurovascular disease A and whose clinical skill level>Care Provider 2 is introduced in this Level 3 (refer FIG. 1 (c))

In this Level using 2 ways interactive audio-video Care provider 3, with the resultant output obtained from the method detailed in FIGS. 1(a) and (b) and findings (NIRS+EEG report, Patient's Medical Information and (X+Y) probability), provides remote diagnosis/screening of neurovascular disease ‘A’ in the Patient to the Care seeker onsite for further clinical management of the Patient.

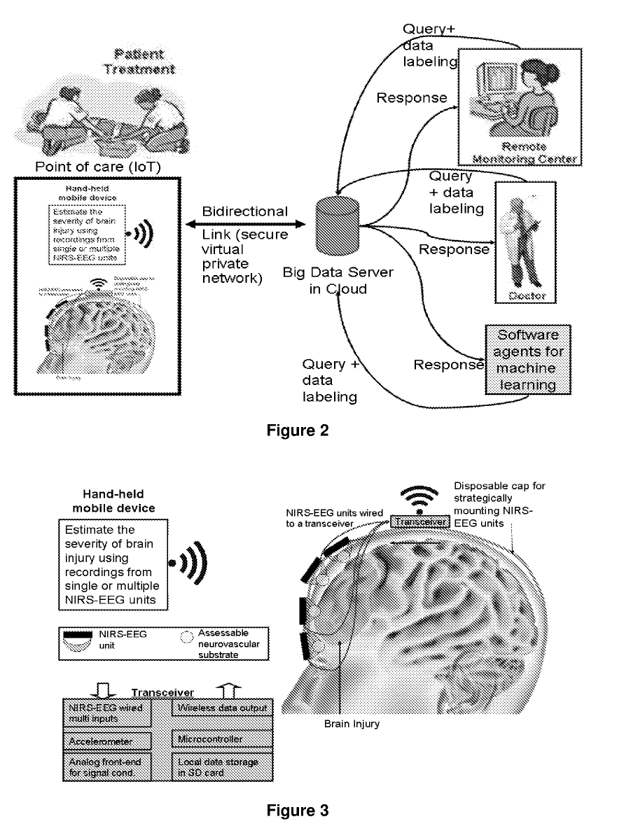

In accordance with another aspect of the present invention, there is provided multiple clients who can interact with the data stored at the Big Data server under different level of authorization. The Software Agent will run machine learning algorithms on the data for classifier validation under query and response. The Software Agent can also create tentative labels and alarms for the data using machine learning algorithms with the lowest confidence level. The Remote Monitoring Human Agent who is a EEG technician can also create tentative labels and alarms for the data with middle confidence level. The neurologist or clinician expert with create labels and alarms for the data with the highest confidence level. This all information will be integrated at the server side with metadata to triage the patient at the PoC, as shown in FIG. 2 that illustrates a high level drawing of a device used for point of care monitoring of neuro-glial-vascular interaction.

In accordance with another aspect of the present invention, there is provided non-invasive detection for neuro-glial-vascular interactions, wherein non-invasive electromagnetic and optical means such as EEG and NIRS (besides blood pressure, PPG, etc.) are used to acquire signals particularly correlated with hemodynamics along with electrical brain measurements, and wherein the changes in EEG during brain trauma are correlated with the changes in NIRS, which enable to measure the state of (inverse) neurovascular coupling and the combined information on dramatic changes in hemodynamics and neuronal activity is integrated to not only monitor (e.g. inverse neurovascular coupling) but assess the outcomes of brain injuries (e.g. deleterious effects of secondary brain injury) (Please refer FIG. 3). Here, NIRS and EEG signal for either the whole brain or a region of interest as well as corresponding sensor location information are sensed by the Transceiver.

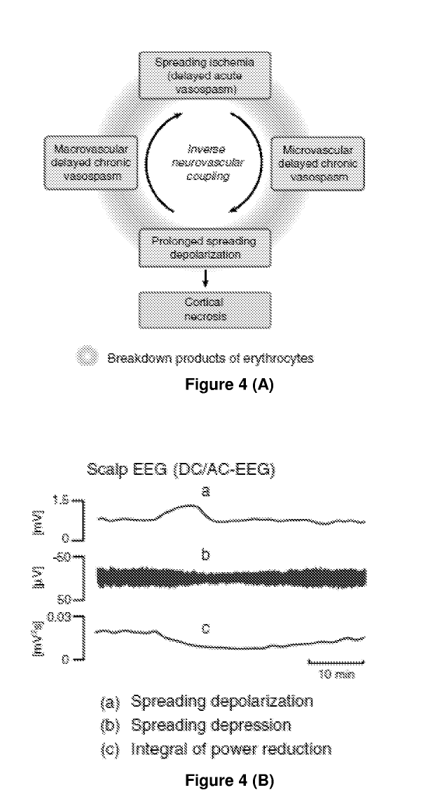

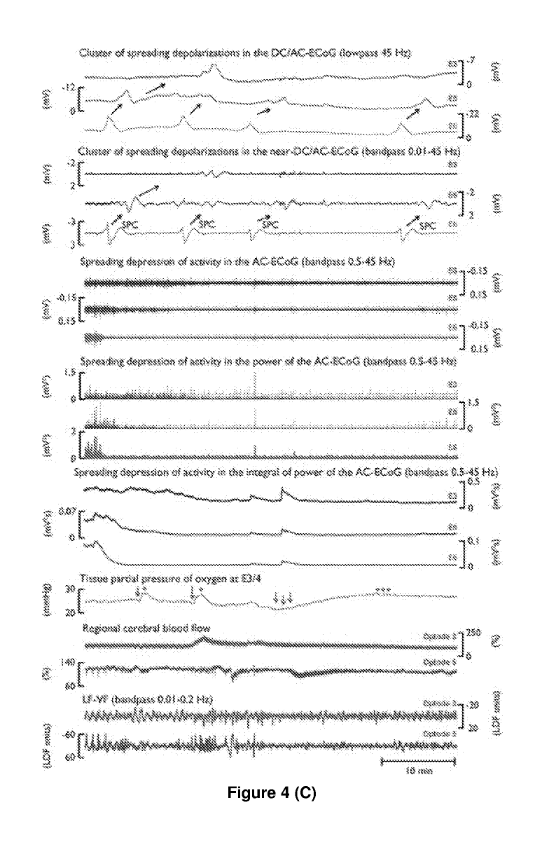

While the above aspects are providing objectives and concepts of the present invention, however, it is anticipated that the invention can be more readily understood through reading the following detailed phenomenological model of the invention and study of the included drawings. An illustrative example is provided in FIG. 4 that shows based on prior works how spreading depolarization can effect both the electromagnetic as well as optical measures of the neural tissue. Here, other conventional systemic measures, e.g. ECG, blood pressure, PPG, etc. are important to build a phenomenological model to determine the signature of the neuro-glial-vascular interaction and dysfunction. The invention will be worked further within this scope and described various aspects while finalizing the complete specification.

According to the present invention, the provided phenomenological model that changes in synaptic transmembrane current resulting in a change in rCBF via a change in the representative radius of the vasculature. Here, it is postulated that the effects of neural activity is elucidated with simultaneous electroencephalography (EEG), which provides an independent measure to supplement NIRS recordings. The complex path from the brain injury-induced change of neural signal recorded with EEG to a change in the concentration of multiple vasoactive agents (such as NO, potassium ions, adenosine), represented by a single vascular flow-inducing vasoactive signal, s, is captured by a first-order Friston's model.

[symbol]

εu(t)−kₛs−g_{f}(f−1)

where f denotes CBF normalized by its baseline value, ε is the neuronal efficacy, kₛ is the rate constant for signal decay, and g_{f} is the gain constant for an auto-regulatory feedback term that drives the CBF back to its baseline value (at steady state: [symbol] 0, s=0 and u(t)=g_{f}(f−1)/ε, i.e., synaptic transmembrane current correlated with baseline-normalized CBF at steady state).

In fact, the intermediate vasoactive agents (such as NO) and metabolic pathways of oxygen utilization (such as cytochrome c oxidase) is selectively stimulated optically thereby facilitating system identification of the NVU. For example, cytochrome c oxidase (Cox) is the primary photoacceptor for the red-NIR range between 630 and 900 nm and either visible (514.5 nm) or long wavelength ultra-violet (lambda=366 nm) light to influence the localized production or release of NO. The released vasoactive signal, s, changes the compliance, C, of the vasculature approximated by first-order kinetics, leading to changes in its representative radius, R, that is captured by a nonlinear compliance model. The photons in the near-infrared (NIR) spectral range (650-950 nm) are able to penetrate human tissue. NIR wavelengths are selected such that the change in concentration of oxy-hemoglobin (HbO2) and deoxy-hemoglobin (Hb) in the brain tissue can be detected. NIRS instrumentation works on different measuring principles, e.g., continuous wave (CW), frequency domain (FD), and time domain (TD). Absolute concentration measurements is possible with more expensive TD and FD techniques, but a relative change in HbO2 and Hb in response to brain injury is all that is necessary for data fitting to estimate neurovascular coupling rather than to quantify the hemodynamic response in absolute terms. For ease in NIRS data fitting, the nonlinear compliance model is linearized about an equilibrium point C_{M}, and the radius, R, was approximated as,

[symbol]

=R≈Rₘₐₓ(1−a₁ exp(−a₂C_{M}))

where Rₘₐₓ is the maximum radius, and a₁ and a₂ are constants. The CBF, i.e., the volume of blood that flows through a unit volume of tissue in a given time unit is approximated using the Ohmic equation,

CBF=K(Pₐ−Pᵥ)R^{γ}

where Pₐ and Pᵥ are arterial and venous blood pressures, K is a constant of proportionality, and the exponent γ is 2 for plug-flow and 4 for laminar flow.

The cerebral metabolic rate of oxygen, CMRO2 (i.e., oxygen consumption), is given by the difference of oxygen flowing into and out of the tissue. CMRO2 is related to CBF as,

$$CMRO 2 = E \cdot {C}_{A} \cdot CBF \\ ⇒ \frac{CMRO 2}{CMRO {2}_{0}} = \frac{E ( f , {E}_{0} )}{{E}_{0}} f$$

where E is the extraction fraction of oxygen (E₀ at baseline). CVR was defined as the ratio between fractional CBF change and fractional CMRO2 change from baseline,

$$CVR = \frac{CMRO 2 / CMRO {2}_{0 }}{f} ⇒ CVR = \frac{E ( f , {E}_{0} )}{{E}_{0}} .$$

The baseline-normalized CMRO2 (i.e. CVR·f) is estimated from baseline-normalized tissue CBF (f), and deoxy- (Hb) and total (Hbt) hemoglobin concentration using the ratio method,

$$( 1 + \frac{CMRO 2}{CMRO {2}_{0}} ) = ( 1 + f ) ( 1 + {γ}_{R} \frac{Hb}{{Hb}_{0}} ) {( 1 + {γ}_{T} \frac{Hbt}{{Hbt}_{0}} )}^{- 1} \\ ⇒ ( 1 + CVR \cdot f ) = ( 1 + f ) ( 1 + {γ}_{R} \frac{Hb}{{Hb}_{0}} ) {( 1 + {γ}_{T} \frac{Hbt}{{Hbt}_{0}} )}^{- 1} \\ ⇒ ( 1 + CVR \cdot {( \frac{Hbt}{{Hbt}_{0}} )}^{γ / 2} ) = ( 1 + {( \frac{Hbt}{{Hbt}_{0}} )}^{γ / 2} ) ( 1 + {γ}_{R} \frac{Hb}{{Hb}_{0}} ) {( 1 + {γ}_{T} \frac{Hbt}{{Hbt}_{0}} )}^{- 1}$$

where the factors γ_{R}ε[0.5,1.5]; γ_{T}ε[0.5,1.5] relate fractional hemoglobin changes in the venous compartment relative to those across all vascular components, and SO2₀ relates oxygen saturation at baseline of the venous compartment to Hbt₀,

$$SO {2}_{0} = \frac{Hb O {2}_{0}}{{Hb}_{0} + Hb O {2}_{0}} ⇒ SO {2}_{0} = \frac{{Hbt}_{0} - {Hb}_{0}}{{Hbt}_{0}} .$$

In case of diffusion-limited oxygen delivery, oxygen consumption is limited by diffusion of oxygen from the vasculature, thus oxygen consumption is tightly coupled to induced blood flow and the surface area of the vasculature (i.e. proportional to R). Here, oxygen utilization following brain injury is probed via the measurement of the oxidation state of cytochrome-c-oxidase using broadband NIRS.

A correlation measure between EEG and NIRS signals may lead to a measure of the state of the neurovascular coupling (NVC), e.g. inverse NVC during spreading depolarizations in brain trauma. If the observed/measured signal don't match the expected healthy signal then there is an abnormality/deficit in the NVU. In an illustrative example, EEG-NIRS based monitoring of NVU, we present a black-box method for the assessment of neurovascular coupling using current source density (CSD) and total hemoglobin concentration estimated from NIRS (Hbt) at the site of brain injury. Empirical Mode Decomposition (EMD) of CSD and Hbt time series into a set of intrinsic mode functions (IMFs) is performed using Huang Hilbert Transform (HHT). Generally, the first IMF contains the highest frequency components and the oscillatory frequencies decrease with increasing IMF index. The IMFs for CSD are denoted as CSDᵢ and IMFs for Hbt are denoted as Hbtᵢ.

The Hilbert transform of an IMF can be denoted as,

$$\begin{matrix}{H}_{CSD , i} ( t ) = \frac{1}{π} P {\int }_{- ∞}^{∞} \frac{{CSD}_{i} ( τ )}{t - τ} d τ & ( 1 ) \\ {H}_{Hbt , i} ( t ) = \frac{1}{π} P {\int }_{- ∞}^{∞} \frac{{Hbt}_{i} ( τ )}{t - τ} d τ & ( 2 )\end{matrix}$$

where P is the Cauchy principal value. Then, the analytic signals are defined as,

Z_{CSD,i}(t)=CSDᵢ(t)+iH_{CSD,i(t)} (3)

Z_{Hbt,i}(t)=Hbtᵢ(t)+iH_{Hbt,i}(t) (4)

The instantaneous amplitudes for the analytic signals can be determined as,

A_{CSD,i}(t)=[CSDᵢ²(t)+H_{CSD,i}²(t)]^{1/2} (5)

A_{Hbt,i}(t)=[Hbtᵢ²(t)+H_{Hbt,i}²(t)]^{1/2} (6)

The instantaneous phases for the analytic signals can be determined as,

$$\begin{matrix}{θ}_{CSD , i} ( t ) = arctan ( \frac{{H}_{CSD , i} ( t )}{{CSD}_{i} ( t )} ) & ( 7 ) \\ {θ}_{Hbt , i} ( t ) = arctan ( \frac{{H}_{Hbt , i} ( t )}{{Hbt}_{i} ( t )} ) & ( 8 )\end{matrix}$$

The instantaneous frequency for the analytic signals can be determined as,

$$\begin{matrix}{f}_{CSD , i} ( t ) = \frac{1}{2 π} \cdot \frac{d {θ}_{CSD , i} ( t )}{dt} & ( 9 ) \\ {f}_{Hbt , i} ( t ) = \frac{1}{2 π} \cdot \frac{d {θ}_{Hbt , i} ( t )}{dt} & ( 10 )\end{matrix}$$

Only the IMFs that had instantaneous frequency less than 11.25 Hz for the whole signal duration were selected for comparison, i.e. cross-spectrum and coherence from 0.5 Hz-11.25 Hz.

The cross-spectrum and coherence between CSD and Hbt can be calculated based on instantaneous amplitude and phase. Here, we will follow a sliding window method where the average instantaneous frequency is first computed. Then, the cross-spectrum at time instant, t, is computed for frequency, fⱼ, from CSD and Hbt from m^{th} and n^{th} observation windows which have average instantaneous frequency closest to fⱼ, i.e.,

C_{f}_{ⱼ}(CSD,Hbt)=A_{CSD,m}(t)A_{Hbt,n}(t)e^{i[θ}^{_{CSD,m}}^{(t)-θ}^{_{Hbt,n}}^{(t)]} (11)

C_{f}_{ⱼ}(Hbt,CSD)=A_{Hbt,m}(t)A_{CSD,n}(t)e^{i[θ}^{_{Hbt,m}}^{(t)-θ}^{_{CSD,n}}^{(t)]} (12)

Also, the coherence is computed as,

$$\begin{matrix}{Coh}_{CSD -> Hbt , {f}_{i}} = \frac{〈 {{C}_{{f}_{j}} ( CSD , Hbt )}^{2} 〉}{〈 {[ {A}_{CSD , m} ( t ) {e}^{i {θ}_{CSD , m} ( t )} ]}^{2} 〉 〈 [ {[ {A}_{Hbt , n} ( t ) {e}^{i {θ}_{Hbt , n} ( t )} ]}^{2} ] 〉} & ( 13 ) \\ {Coh}_{Hbt -> CSD , {f}_{j}} = \frac{〈 {{C}_{{f}_{j}} ( Hbt , CSD )}^{2} 〉}{〈 {[ {A}_{Hbt , m} ( t ) {e}^{i {θ}_{Hbt , m} ( t )} ]}^{2} 〉 〈 [ {[ {A}_{CSD , n} ( t ) {e}^{i {θ}_{CSD , n} ( t )} ]}^{2} ] 〉} & ( 14 )\end{matrix}$$

where [symbol] denotes averaging over multiple paired windows for the given frequency, fⱼ. Here, significant positive values point to a causal relation.

The neurovascular coupling (NVC) for the given frequency, fⱼ can be estimated from cross-spectral power and coherence as,

NVC(fⱼ)= [symbol] C_{f}_{ⱼ}(CSD,HBt)² [symbol] Coh_{f}_{ⱼ} (15)

From this NVC spectrogram using brain injury evoked neuronal and hemodynamic responses, the degree of NVC at a certain time can be assessed based on the sum of power in a frequency band of interest, e.g., Theta band or Alpha band. Furthermore, such markers derived from NIRS-EEG for neurovascular disorders need to be established first from population studies. Burst suppression, in which bursts of electrical activity alternate with periods of quiescence or suppression is a well-known, readily discernible EEG marker of profound brain inactivation and unconsciousness. This pattern is commonly maintained when anesthetics are administered to produce a medically-induced coma for cerebral protection in patients suffering from brain injuries or to arrest brain activity in patients having uncontrollable seizures.

According to the present invention, it is postulated that system identification techniques, e.g., an autoregressive (ARX) model is applied to capture the coupling relation between IMFs of regional cerebral hemoglobin oxygen saturation and the log-transformed mean-power time-series of IMFs for EEG from the lesional and contralesional hemispheres. Subject-specific alterations of ARX poles and zeros with different dead time is relevant for diagnosing neurovascular dysfunction. These algorithms are computationally expensive so we are prototyping field programmable specialized electronic circuit to rapidly manipulate and alter memory to accelerate the computation that can eventually go in application-specific integrated circuit.

The linear time variant system can be described by an autoregressive model with exogenous input (ARX), which has been shown experimentally to yield good tracking of output NIRS signal, given EEG as the input.

It can be described as

A(z)y(t)=B(z)u(t)+e(t) (1)

with transfer function

$$G ( z ) = \frac{B ( z )}{A ( z )}$$

and

A(z)=1+a₁z⁻¹+1+a₂z⁻²+ . . . +1+a₁z⁻¹

B(z)=b₁z⁻ⁿ+1+b₂z⁻⁽¹⁺ⁿ⁾+ . . . +1+bₘz^{-(n+m-1)} (2)

where y(t) is the output and u(t) is the input at any time t. The z−1 is a back shift operator and (z−1) y(t) is equal to y(t−1). e(t) is the zero mean and gaussian white noise affecting the system. The model has l+m parameters/coefficients in total (a1 . . . al, b1 . . . bm).

Substituting (2) in (1), and expanding, the output of an ARX model can be parameterized as

$$y ( t , θ ) = {\sum }_{i = 1}^{l} {a}_{i} y ( t - i ) + {\sum }_{j = 1}^{m} {b}_{j} u ( t + n - j ) \qquad ( 3 )$$

where θ=(a1 . . . al, b1 . . . bm). The size of θ depends on complexity of the model. Thus, the selection of model order (l,m,n) becomes a crucial step in the estimation of unknown parameters in θ.

The elements of θ are time varying as it relates to variation in EEG power to NIRS response. At a given time t, the model estimates are predicted using equation (3), assuming that the system is stationary (slowly time varying), during the prediction horizon. Considering an ARX (l,m,n) model as described in equation (3), its space state form can be described as:
1. Process Equation:

xₖ=Axₖ₋₁+Buₖ₋₁ (4)
2. Measurement Equation:

yₖ=Cxₖ (5)

where k represents the current time step. In equation (4), the current state vector xk=[x1 . . . xq] where q=max(l,m) and uk−1 is the previous model input.

A∈R (q×q) matrix relates the previous state xk−1 to the current state xk. B∈R (q×1) matrix relates the previous input uk−1 to the current state xk.

$$A = [ \begin{matrix}a 1 & 1 & 0 & … & … & 0 \\ a 2 & 0 & … & … & … & 0 \\ … & … & … & … & … & … \\ … & … & … & … & … & … \\ aq - 1 & 0 & 0 & … & … & 1 \\ aq & 0 & 0 & … & … & 0\end{matrix} ] , B = [ \begin{matrix}{u}_{1} \\ {u}_{2} \\ … \\ … \\ uq - 1 \\ uq\end{matrix} ]$$

The yk in equation (5) is the measurement of system output. C∈R (1×q) matrix relates the current state to current measurement with the following expression:

C=[1 0 0 . . . 0 0].

Matrices A, B, C might change with each time-step or measurement, but in this study we assume that they are constant for simplification.

In accordance with another exemplary embodiments of the present invention, there is provided non-invasive detection for hypoxic ischemic encephalopathy (HIE) in neonates and children, wherein non-invasive optical means such as EEG and NIRS are used to acquire signals particularly correlated with hemodynamics along with electrical brain measurements, and wherein the changes in EEG during brain ischemia are correlated with the changes in NIRS, which enable to measure the state of neurovascular coupling and the combined information on dramatic changes in hemodynamics and neuronal activity is integrated to not only monitor or assess the outcomes of ischemia (e.g. deleterious effects of secondary brain injury), but also to monitor and guide therapeutic interventions like hypothermia.

Cerebral perfusion and oxygenation are key biomarkers of brain metabolism and may be disrupted in neonates with HIE. Near-infrared spectroscopy (NIRS) provides reproducible, quantitative measures of cerebral blood volume (reflecting perfusion) and regional oxygen saturation (rSO2), which could represent cerebral metabolism. In addition, NIRS also records regional mixed venous saturation (SctO2), which are representative of oxygen supply/demand ratio. NIRS monitoring easily allows serial measures to be taken over time, which may be highly valuable in disorders such as HIE where brain perfusion and oxygen metabolism change over the course of the illness. Also, Amplitude-integrated EEG (aEEG) has prognostic value in the first hours after neonatal asphyxia.

In one embodiment, the invention provides a method of identifying risk of HIE in a distressed neonate comprising a step of collecting EEG and NIRS signal obtained from the distressed neonate, wherein an rSO2 and SctO2 values are (rScO2 values were significantly higher in this group as compared with the favorable outcome group at 24, 36, 48, and 84 h postnatally (mean±SD: 82±7 vs. 72±9%, 83±9 vs. 75±8%, 83±10 vs. 76±8%, 79±10 vs. 72±9%, P<0.001, P<0.01, P<0.05, and P<0.02, respectively http://www.nature.com/pr/journal/v74/n2/full/pr201384a.html) and aEEG (The median upper margin of the widest band of aEEG activity in the control infants was 37.5 microV (range, 30-48 microV), and median lower margin was 8 microV (range, 6.5-11 microV). We classified the aEEG background activity as normal amplitude, the upper margin of band of aEEG activity >10 microV and the lower margin >5 microV; moderately abnormal amplitude, the upper margin of band of aEEG activity >10 microV and the lower margin <1=5 microV; and suppressed amplitude, the upper margin of the band of aEEG activity <10 microV and lower margin <5 microV. Recordings were analyzed further for the presence of seizures, defined as periods of sudden increase in voltage accompanied by a narrowing of the band of aEEG activity. Tests of interobserver variability showed excellent agreement both for assessment of amplitude (kappa statistic=0.85) and for identification of seizures (kappa statistic=0.76) There was a close relationship between the aEEG and subsequent outcome: 19 of 21 infants with a normal aEEG finding were normal on follow-up at 18 to 24 months of age, whereas 27 of 35 infants with a moderately abnormal or suppressed aEEG and/or seizures died or developed neurologic abnormalities. Thus, aEEG predicted outcome with a sensitivity of 0.93, a specificity of 0.70, positive predictive value of 0.77, negative predictive value of 0.90, and the likelihood ratio of a positive result of 3.1 and a negative result of 0.06. For the 24 infants studied within 12 hours of birth, the corresponding results were sensitivity, 1.0; specificity, 0.82; positive predictive value, 0.85; negative predictive value, 1; likelihood ratio of a positive result, 5.5; and likelihood ratio of a negative result, 0.18. https://www.ncbi.nlm.nih.gov/pubmed/10353940) indicative of a risk of HIE in the neonate. “Of 39 infants, 12 neonates died because of neurological deterioration. One had an adverse outcome and 26 had a favorable outcome. The rScO2 was higher in neonates with adverse outcome, although aEEG scores were lower. Positive predictive values at 12, 24, and 36 h of age for adverse outcome ranged from 50 to 67% for rScO2 and aEEG; negative predictive values ranged from 73 to 96% for rScO2 and 90 to 100% for aEEG. Combining rScO2 and a EEG increased positive predictive values (70-91%) and negative predictive values (90-100%).

The invention provides a method of treating a neonate at risk of having HIE (or severe HIE), the method comprising the steps of:

(a) identifying a neonate at risk of having HIE (or severe HIE) according to a method of the invention, and

(b) treating the neonate identified as being at risk of having HIE (or severe HIE) in step (a) with a neuroprotective therapy.

The invention provides a method of treating a neonate identified as having a risk of having HIE (or severe HIE) according to a method of the invention, the method comprising the steps of treating the neonate identified as being at risk of having HIE (or severe HIE) with a neuroprotective therapy.

The invention also provides a system for determining whether a neonate is at risk of having or developing HIE, the system comprising:

(a) a Detection module configured to receive NIRS and EEG signal for either the whole brain or a region of interest as well as the location information of those sensors (i.e., sensor montage using cap with shape detection capability), and perform analysis (e.g., ARX method),

(b) a Storage module for locally storing data relating to NIRS and EEG sensors, e.g., sensor sensitivity values, such that the NIRS and EEG sensors can be re-configured (i.e., change in the sensor montage) to focus on a region of interest for the Detection module;

(c) a Processing module for comparing the detected abnormalities in NIRS and EEG values with a reference signal values, e.g., that are stored in the Storage module, as well as to determine the sensor montage to target as queried region of interest according to sensor sensitivities stored in Storage module;

(d) a Display module for displaying a content based in part on the data output from said Detection module, wherein the content comprises a signal indicative of the presence or absence and/or severity of the neuro-glial-vascular dysfunction.

(e) a Transfer module to bidirectionally transfer the data wirelessly to a remote monitoring center (e.g., over a secure virtual private network) and for synchronizing streaming data for live analysis or recording

The term “hypoxic-ischaemic encephalopathy” or “HIE” describes the brain insult which results from insufficient oxygen or blood supply to the newborn brain during labour and delivery, and consequent brain pathology, brain swelling or later brain injury. As employed herein, the term HIE should be understood to encompass mild, moderate or severe HIE. Moderate and severe HIE are characterized by lethargy, hypotonia, diminished deep tendon reflexes, occasional periods of apnoea, seizures, and absence of grasping, Moro, and sucking reflexes. Typically, the term HIE should be understood to include neonatal encephalopathy.

All directional and numeral references used herein are only used for identification purposes to aid the reader's understanding of the present invention, and do not create limitations, particularly as to the position, orientation, or use of the invention.

It will thus be seen that the objects set forth above, among those made apparent from the preceding description, are efficiently attained and, since certain changes may be made in the above system without departing from the spirit and scope of the invention, it is intended that all matter contained in the above description and shown in the accompanying drawings shall be interpreted as illustrative and not in a limiting sense.

It is also to be understood that the following claims are intended to cover all of the generic and specific features of the invention herein described and all statements of the scope of the invention which, as a matter of language, might be said to fall there between.

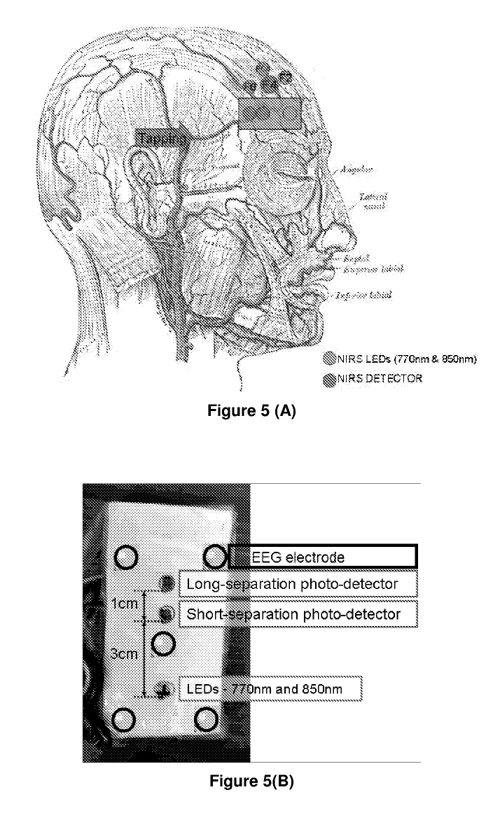

In accordance with the method provided herein, anterior temporal artery tap (FIG. 5A) is used to identify systemic interference using short-separation NIRS measurements (FIG. 5B) during the monitoring which enable to remove the systemic interference occurring in the superficial layers of the head during the monitoring.

In accordance with the method provided herein, the human and/or software agents can remotely query (see FIG. 2) a specific neural tissue or cortical location (region of interest) for whole-head montage of EEG and NIRS sensors at the point of care (POC) (see FIG. 2); and the sensor montage will automatically reconfigure at the POC device based on pre-computed (and stored) sensitivity of the sensor locations and the sensor cap with shape detection capability. The shape detection can be based on prior works on 2D impedance mapping (e.g. bend sensing technology: https://www.ncbi.nlm.nih.gov/pmc/articles/PMC4970015/).

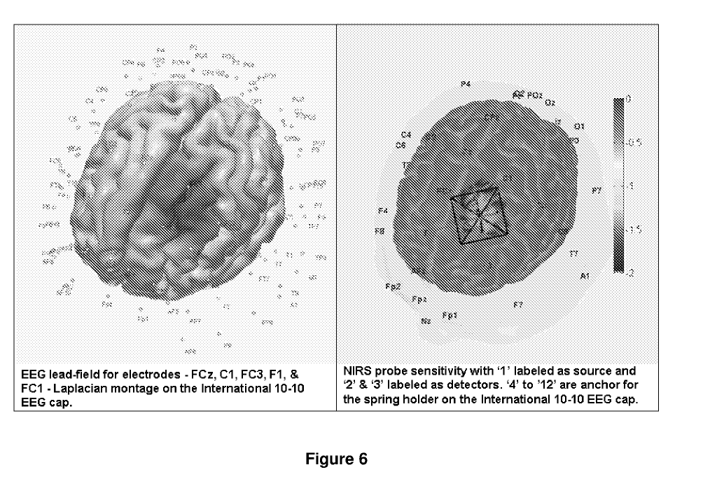

FIG. 6 illustrates an illustrative NIRS-EEG joint-imaging of same brain tissue primarily in the middle frontal and superior frontal gyrus of brodmann area 6 to assess neurovascular coupling in that region of interest.

The foregoing has outlined features of several embodiments so that those skilled in the art may better understand the present disclosure. Those skilled in the art will also appreciate that the present disclosure may be used as a basis for designing or modifying other processes and structures for carrying out the same purposes and/or achieving the same advantages of the embodiments introduced herein. Those skilled in the art should also realize that such equivalent constructions do not depart from the scope of the present disclosure, and that they may make various changes, substitutions and alterations herein without departing from the scope of the present disclosure, as defined by the following claims.

1-26. (canceled)

27. A bi-directional, decision-tree based Point of Care (POC) system, the system comprising:
Point of Care (POC) device(s) that comprising one or more sensors capable to acquire multiple bio-signals from brain and/or body within a body area network;
a Detection module configured to receive signal for either the whole brain or a region of interest as well as corresponding sensor location information and perform analysis;
an Electronics/electrical component(s) of the device configured to capture one or more bio-signals from the sensors as distinct but synchronized signals;
a Storage for locally storing data relating to the sensors such that the sensors are reconfigured to focus on a region of interest for the Detection module;
a Processing module for comparing the detected abnormalities in the analysis values with a reference signal values;
a Display module for displaying a content based in part on the data output from said Detection module, wherein the content comprises a signal indicative of the presence or absence and/or severity of the neuro-glial-vascular dysfunction;
a Transfer module to bidirectionally transfer the data wirelessly to a remote monitoring center and for synchronizing streaming data for live analysis or recording
an internet of things enabled component with a Graphical User Interface (GUI) of the device interacting with the electronics/electrical component wirelessly or in a wired manner to receive the bio-signals as well as captures patient's vitals and other pertinent medical information from multiple sources and transfers the bio-signals along with the patient's medical information to a tele-monitoring platform; and
a multi-level, decision-tree based diagnosis and triaging deployed in the telemonitoring platform analyzes the “input”, aggregates the output decision at each level of the decision tree and provides the output decision as input to next level of the decision-tree system leading towards accurate diagnosis of patient's medical condition.

28. The system of claim 27, wherein the one or more sensors comprises electroencephalography (EEG) and/or near-infrared spectroscopy (NIRS) as well as extended near infrared spectroscopy from ˜700 nm to 2500 nm for multi-distance optical monitoring and/or tomography of cerebral tissue.

29. The system of claim 27, wherein the one or more sensors performs simultaneous multi-modality multi-distance recording targeting the same neural tissue.

30. The system of claim 27, wherein the POC device contains audio video capabilities.

31. The system of claim 27, wherein the POC device transmits one or more clinical parameters to remote care providers and the remote care providers use audio video capabilities of the POC device to observe and interact with the patient for diagnosing patient's condition in the Diagnosis module.

32. The system of claim 31, wherein the remote care providers have different levels of expertise such that the Diagnosis module incorporates their corresponding decision confidence information.

33. The system of claim 27, wherein the one or more parameters comprises neurological conditions sensed by the one or more sensors, physiological observations provided by a care seeker and output of multi-level Diagnosis module.

34. The system of claim 27, wherein the POC system runs along with artificial intelligence and machine learning algorithms within the Diagnosis module.

35. A Point of Care (POC) system for determining neuro-glial-vascular interaction, wherein the system comprising:
one or more sensor (re)configured to sense a particular characteristic indicative of a neurological or psychiatric condition or state;
means for receiving an input from one or more remotely configurable sensors to target a cortical region of interest, and developing treatment parameters based on the input data;
a determination means configured to receive NIRS and EEG signal and perform analysis remotely;
means for storing and comparing the data relating to NIRS and EEG signal generated by the determination means for comparing the detected abnormalities in NIRS and EEG values with a reference signal values;
means for the human and/or software agents to remotely query a specific neural tissue or cortical location (region of interest) from the whole-head distribution of EEG and NIRS sensors at the point of care (POC);
means of finding the subject specific shape of the cap, e.g., based on the impedance changes in the cap material, and therefore the EEG and NIRS sensor locations such that the sensor montage can automatically re-configured from the whole-head distribution at the POC device based on pre-computed sensitivity function of the sensor locations;
a display means for displaying a content based in part on the data output from said determination means, wherein the content comprises a signal indicative of the presence or absence and/or severity and a transfer means to transfer the data wirelessly to a remote monitoring center to take the required decision with the Diagnosis module,
wherein the system characterized in that utilizes two or more sensor modalities such as multi-wavelength optical and electrophysiological, which are used simultaneously to target same neural substrate in a single sensor montage using beam-forming approaches to enables effective point of care monitoring of the neurological disorders and neurovascular diseases.

36. The system as claimed in claim 35, wherein the simultaneous multi-modality multidistance recording of EEG and multi-wavelength multi-distance NIRS are enable to sense brain activity by targeting same neural substrate simultaneously and passing the signals to the analyzer and wherein said analyzer analyzes the brain activity for any neurological disorders and neurovascular diseases and adds to the therapeutic accessibility of the disorder and disease under a remote human-in-loop triage decision making system.

37. The system as claimed in claim 35, wherein said system is point of care multi-modal and enable real time continuous functioning remotely.

38. A point-of-care monitoring (POCT) method for neuro-glial-vascular interactions, wherein the method comprising:
receiving an input of a neurological or psychiatric condition from one or more sensors and developing treatment parameters based on the input data,
determining the signals received from electroencephalography (EEG) is used with Near Infrared spectroscopy (NIRS) and comparing the data relating to NIRS and EEG signal for the detected abnormalities values with a reference signal values,
further analyzing and transferring the data wirelessly to a remote monitoring center to take the required decision,
wherein the method characterized in that utilizes two or more sensor modalities such as multi-wavelength optical and electrophysiological, which are used simultaneously to target same neural substrate in a single sensor montage using beam-forming approaches to enables effective point of care monitoring of the neurological disorders and neurovascular diseases.

39. The method according to claim 38, wherein the communication is bidirectional between the POC device and the remote human as well as software agents working in concert and wherein point of care (POC) data as well as metadata (observations by paramedic) is relayed by the device client (IoT) to remote telemonitoring center (data server) where it's tagged online from synchronized streaming data for live analysis or recording for neurovascular dysfunction and the NIRS-EEG sensor montage is automatically reconfigured at the server side to target that specific neural tissue at POC.

40. The method as claimed in claim 38, wherein the one or more sensors are enable to sense brain activity by targeting same neural substrate simultaneously and passing the signals to the analyzer and wherein said analyzer analyzes the brain activity from synchronized streaming data for live analysis or recording for any neurological disorders and neurovascular diseases.

41. The method as claimed in claim 38, wherein the one or more sensors comprise electroencephalography (EEG) and near-infrared spectroscopy (NIRS) along with other analytical tool, wherein said EEG and NIRS are used simultaneous from synchronized streaming data for live analysis or recording to detect spreading depolarization in brain trauma.

42. The method as claimed in claim 38, wherein the simultaneous multi modality multidistance recording of EEG and multi-wavelength NIRS during spreading depolarization/depression of spontaneous activity is not only detect NVC dysfunction and assess secondary brain injuries, but also adds to the therapeutic accessibility of the syndrome under a remote human-in-loop triage decision making system.

43. The method according to claim 38, wherein the system comprising integrating the POCT device for brain trauma monitoring from synchronized streaming data for live analysis or recording at the Medical Emergency System towards point-of-care sensors with remote human-in-loop triage decision making system using internet of things.

44. The method according to claim 38, wherein the sensor enable interaction between the different components used herein in the system such as data from synchronized streaming data for live analysis or recording is received and spread or bifurcated with Doctor and Data analytics center and wherein said analytics center provides diagnosis of neurovascular dysfunction in cerebrovascular occlusive disease of the patient.

45. The method as claimed in claim 38, wherein said method is point of care multi-modal and enable real time continuous functioning remotely from synchronized streaming data for live analysis or recording.

46. The method as claimed in claim 38, wherein said method is autoregressive (ARX) method and wherein said method is utilized to capture the coupling relation between regional cerebral haemoglobin oxygen saturation and the log-transformed mean-power time-series for EEG, wherein subject-specific alterations of ARX poles and zeros with different dead time provides relevance for diagnosing neurovascular dysfunction from synchronized streaming data for live analysis or recording in cerebrovascular occlusive disease.
